(19) United States
(12) Patent Application Publication
(10) Pub. No.: US 2022/0177978 A1
(43) Pub. Date: Jun. 9, 2022

(54) METHODS OF PREDICTING AND PREVENTING CANCER IN PATIENTS HAVING PREMALIGNANT LESIONS

(71) Applicants: INSERM (Institut National de la Santé et de la Recherche Médicale), Paris (FR); Université de Paris, Paris (FR); Sorbonne Université, Paris (FR); Université d'Aix Marseille, Marseille Cedex 07 (FR); Assistance Publique-Hôpitaux de Marseille, Marseille (FR); Université Libre de Bruxelles, Bruxelles (BE); Trustees of Boston University, Boston, MA (US)

(72) Inventors: Jérôme GALON, Paris (FR); Céline MASCAUX, Strassbourg (FR); Mihaela ANGELOVA, Paris (FR); Jean-Paul SCULIER, Brussels (BE); Jennifer BALE, Boston, MA (US); Kahkeshan HIJAZI, Boston, MA (US); Avurm SPIRA, Boston, MA (US)

(21) Appl. No.: 17/600,957

(22) PCT Filed: Apr. 1, 2020
(86) PCT No.: PCT/EP2020/059272

§ 371 Date: Oct. 1, 2021

(30) Foreign Application Priority Data
Apr. 2, 2019 (EP) 19305434.3
Apr. 26, 2019 (EP) 19305535.7

(51) Int. Cl. C12Q 1/6886; A61K 39/00; A61K 45/06; A61P 35/00

(52) U.S. Cl. CPC C12Q 1/6886; A61K 39/0005; A61K 45/06; C12Q 2600/118; C12Q 2600/158; C12Q 2600/156; A61P 35/00

(57) ABSTRACT

As advanced cancer has poor prognosis, its detection and treatment at the earliest stages is critical to increase cancer survival rate. Therefore, elucidating the determinants of the intra-lesion immune reaction during cancer's developments is critical for moving into precision medicine and immunotherapy-based cancer prevention. Adaptive immune response within tumors was shown to be the strongest at the earliest stage of carcinoma. Thus, the inventors hypothesized that the immune microenvironment and adaptive immunity were first established at early stage of lung carcinogenesis. Here they identified changes in the tumor molecular profile and its microenvironment during the successive steps of lung squamous carcinogenesis, using gene expression profiling and multispectral imaging. A unique and invaluable dataset of (9) morphological stages of development was analyzed, including (122) well-annotated biopsies from (77) patients. In particular, the inventors show that immune activation and immune escape occur before tumor invasion, and that immunosuppressive cytokines and checkpoint receptors immune escape mechanisms are concomitant with anti-tumor immunity in high-grade dysplasia. Thus, the present invention relates to methods of predicting and preventing cancer in subjects having premalignant lesions.

### FIELD OF THE INVENTION

The field of the invention is oncology and immunology.

### BACKGROUND OF THE INVENTION

Early intervention in cancer's development stage is the only opportunity to cure subjects from cancer up to now. Among early intervention, the most promising include early detection, if possible at precancerous stage even before invasion occurs, allowing for radical and curative resection of the tumor, combined with neoadjuvant or adjuvant treatment to reduce the risk of relapse related to micrometastases. The other possible early intervention to reduce cancer's burden is the prevention of cancer, including primary prevention as smoking cessation in example, and also secondary prevention with the emergence of chemoprevention approaches. In order to detect or prevent cancer at the earliest stage, the study of the molecular mechanisms involved in premalignant lesions and their microenvironment is required. Premalignant lesion is a morphologically altered tissue in which cancer is more likely to occur than its apparently normal counterpart. These include among others leukoplakia, erythroplakia, and the palatal lesions of reverse smokers, barrets esophagus, and adenomatous polyps of stomach or colon. For instance, smoking exposes the respiratory mucosa to carcinogens leading to a “field cancerization” process. Smokers develop a range of successive pre-invasive stages preceding the development of invasive lung cancer, which characterize this multistep evolutionary process. Following the development of fluorescence bronchoscopy, lung squamous pre-invasive lesions can be collected and studied. However, despite the development of technological advances, the rarity of pre-invasive lesions collections explains the limited knowledge of their molecular and immune profile.

### SUMMARY OF THE INVENTION

As defined by the claims, the present invention relates to methods of detecting, predicting and preventing cancer with a prophylactic treatment in subjects having premalignant lesions.

### DETAILED DESCRIPTION OF THE INVENTION

As advanced cancer has poor prognosis, its detection and treatment at the earliest stages is critical to increase cancer survival rate¹. Therefore, deciphering the molecular processes arising in premalignant lesions and the role of their microenvironment is critical for better understanding of the biology behind carcinogenesis. Elucidating the determinants of the intra-lesion immune reaction during cancer's developments is critical for moving into precision medicine and immunotherapy-based cancer prevention². Adaptive immune response within tumors was shown to be the strongest at the earliest stage of carcinoma³. Thus, the inventors hypothesized that the immune microenvironment and adaptive immunity were first established at early stage of lung carcinogenesis. Here they identified changes in the tumor molecular profile and its microenvironment during the successive steps of lung squamous carcinogenesis, using gene expression profiling and multispectral imaging. A unique and invaluable dataset of 9 morphological stages of development was analyzed, including 122 well-annotated biopsies from 77 patients. The results delineate the sequential molecular pathways as follow 1) linearly form normal tissue to cancer, a continuous increase of proliferation and DNA repair; 2) a transitory increase in low-grade pre-invasive lesion of metabolism and early immune sensing by activation of resident immune cells and acquisition of memory phenotype; 3) from high-grade pre-invasive lesion, the activation of immune response and immune escape (including immune checkpoints PDL1, PD1, IDO1, CTLA4, TIGIT, and suppressive interleukins, including IL6, IL10, and TGFβ), and 4) ultimately at the invasive stage, activation of the epithelial-mesenchymal transition (EMT), including CXCR4. The inventors show that immune activation and immune escape occur before tumor invasion, and that immunosuppressive cytokines and checkpoint receptors immune escape mechanisms are concomitant with anti-tumor immunity in high-grade dysplasia.

These data support the detection of immune-based early biomarkers and the potential use of immunotherapy for chemopreventive approaches for individuals at high-risk of developing cancer.

General Inventive Concept of the Present Invention:

Thus, the present invention relates to a method for determining whether a subject having a premalignant lesion is at risk of having a cancer comprising determining the level of at least one immune marker in a biological sample obtained from the subject and wherein the expression level of the immune marker correlates with the risk of having cancer.

As used herein, the term “premalignant lesion” means tissue that is not yet malignant, but may be capable of becoming malignant. As used herein, the terms “lesion” refer to an area of a tissue that has, or appears to have, undergone a pathological change. For example, a premalignant lesion may be histologically identified as metaplastic, hyperplastic, dysplastic or an in situ carcinoma. In some embodiments, the premalignant lesion is a low or high grade dysplasia. Dysplasia is defined as an unequivocal neoplastic alteration of the epithelium. Dysplasia can itself be subdivided objectively into high grade and low grade depending on the proportion of dysplastic cells in the epithelium. In low grade dysplastic cells are largely confined to the basal layers of the epithelium, whereas in high grade dysplasia they regularly reach the upper part of the epithelium.

In some embodiments, the subject follows a surveillance program. As used herein, the term “surveillance program” refers to a set of examinations or procedures used to longitudinally follow up individuals identified in a screening program to have premalignant lesions. A “surveillance program” includes strategies for both surveillance interval and surveillance intensity. For instance, examination may be performed by one or more suitable procedures, e.g., endoscopy (e.g. bronchoscopy, colonoscopy and sigmoidoscopy), sample occult blood testing, computed tomography (CT) or other imaging procedure.

In some embodiments, the method of the present invention is particularly suitable for predicting the risk of having a cancer that results from polygenic or multifactorial phenotypes. In some embodiments, the method of the present invention is particularly suitable for predicting the risk of cancer in a subject exposed or previously exposed to exogenous factors such as sun, tobacco, alcohol, pollution, certain chemical, or radiation.

In some embodiments, the method of the present invention is particularly suitable for predicting a risk of a cancer selected from the group consisting of adrenal cortical cancer, anal cancer, bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, Chorioadenoma destruens), Hodgkin's disease, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer, squamous lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma,), vaginal cancer, vulvar cancer, and uterine cancer (e.g. uterine leiomyosarcoma).

In some embodiments, the method of the present invention is particularly suitable for predicting the risk of having a lung cancer.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

Samples:

As used herein, the term “sample” to any biological sample obtained from the purpose of evaluation in vitro.

In some embodiments, the biological sample is a body fluid sample. Examples of body fluids are blood, serum, plasma, amniotic fluid, brain/spinal cord fluid, liquor, cerebrospinal fluid, sputum, throat and pharynx secretions and other mucous membrane secretions, synovial fluids, ascites, tear fluid, lymph fluid and urine. More particularly, the sample is a blood sample. As used herein, the term “blood sample” means a whole blood sample obtained from the patient.

In some embodiments, the biological sample is a tissue sample. The term “tissue sample” includes sections of tissues such as biopsy or autopsy samples and frozen sections taken for histological purposes. In some embodiments, the tissue sample obtained from the premalignant lesion. Said tissue sample is obtained for the purpose of the in vitro evaluation. In some embodiments, the tissue sample may result from a biopsy performed in the premalignant lesion of the patient.

Immune Markers:

As used herein, the term “immune marker” consists of any detectable, measurable or quantifiable parameter that is indicative of the status of the immune response of the subject.

In some embodiments, the immune marker includes the presence of, or the number or density of, cells from the immune system. In some embodiments, the immune marker includes the presence of, or the amount of proteins specifically produced by cells from the immune system. In some embodiments, the immune marker includes the presence of or the amount of the proteins that are released as soluble form (e.g. in a body fluid such as blood). In some embodiments, the immune marker includes the presence of, or the amount of, any biological material that is indicative of the level of genes related to the raising of a specific immune response of the host. Thus, in some embodiments, the immune marker includes the presence of, or the amount of, messenger RNA (mRNA) transcribed from genomic DNA encoding proteins which are specifically produced by cells from the immune system. In some embodiments, the immune marker includes surface antigens that are specifically expressed by cells from the immune system, including by B lymphocytes, T lymphocytes, monocytes/macrophages dendritic cells, NK cells, NKT cells, and NK-DC cells or alternatively mRNA encoding for said surface antigens.

An immune marker becomes an “immune marker” for the purpose of carrying the method of the present invention when a good statistical correlation is found between (i) an increase or a decrease of the quantification value for said marker and (ii) the occurrence of cancer. For calculating correlation values for each marker tested and thus determining the statistical relevance of said marker as a “immune marker” according to the invention, any one of the statistical method known by the one skilled in the art may be used. Illustratively, statistical methods using univariate analysis using the log-rank-test and/or a Cox proportional-hazards model may be used, as it is shown in the examples herein. Any marker for which a P value of less than 0.05, and even preferably less than 10⁻³, 10⁻⁴, 10⁻⁵, 10⁻⁶ or 10⁻⁷ (according to univariate and multivariate analysis (for example, log-rank test and Cox test, respectively) is determined consists of a “immune marker” useable in the method of the invention. When performing method of the present invention with more than one immune marker, the number of distinct immune markers that are quantified at step a) are usually of less than 100 distinct markers, and in most embodiments of less than 50 distinct markers. The number of distinct immune markers that is necessary for obtaining an accurate and reliable prognosis, using the method of the present invention, may vary notably according to the type of technique for quantification. Illustratively, high statistical significance can be found with a combination of a small number of immune markers, when the method of the present invention is performed by in situ immunohistochemical detection of protein markers of interest. In some embodiments, the level of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50 markers is (are) determined.

In the present specification, the name of each of the various immune markers of interest refers to the internationally recognised name of the corresponding gene, as found in internationally recognised gene sequences and protein sequences databases, including in the database from the HUGO Gene Nomenclature Committee that is available notably at the following Internet address: http://www.gene.ucl.ac.uk/nomenclature/index.html. In the present specification, the name of each of the various immune markers of interest may also refer to the internationally recognised name of the corresponding gene, as found in the internationally recognised gene sequences and protein sequences database Genbank. Through these internationally recognised sequence databases, the nucleic acid and the amino acid sequences corresponding to each of the immune marker of interest described herein may be retrieved by the one skilled in the art.

Methods for Determining Whether a Subject Having a Low Grade Dysplasia is at Risk of Having a Cancer:

In some embodiments, the present invention relates to a method for determining whether a subject having a low grade dysplasia is at risk of having a cancer comprising determining the level of at least one immune marker in a biological sample obtained from the subject wherein the immune marker is selected from the group consisting of CD58 and SERPIN members and wherein the expression level of the immune marker correlates with the risk of having cancer.

More particularly, the present invention relates to a method for determining whether a subject having a low grade bronchial dysplasia is at risk of having a lung cancer comprising determining the level of at least one immune marker in a biological sample obtained from the subject wherein the immune marker is selected from the group consisting of CD58 and SERPIN members and wherein the expression level of the immune marker correlates with the risk of having cancer.

As used herein, the term “CD58” has its general meaning in the art and refers to the lymphocyte function-associated antigen 3 (LFA-3) that is a cell adhesion molecule expressed on Antigen Presenting Cells (APC), particularly on macrophages (Barbosa J A, Mentzer S J, Kamarck M E, Hart J, Biro P A, Strominger J L, Burakoff S J (April 1986). “Gene mapping and somatic cell hybrid analysis of the role of human lymphocyte function-associated antigen-3 (LFA-3) in CTL-target cell interactions”. J. Immunol. 136 (8): 3085-91.; Wallich R, Brenner C, Brand Y, Roux M, Reister M, Meuer S (15 Mar. 1998). “Gene structure, promoter characterization, and basis for alternative mRNA splicing of the human CD58 gene”. J. Immunol. 160 (6): 2862-71).

As used herein, the term “SERPIN members” are a superfamily of proteins with similar structures that were first identified for their protease inhibition activity. Protease inhibition by serpins controls an array of biological processes, including inflammation. Examples of SERPIN members include Angiotensinogen, Antithrombin-III, Leukocyte elastase inhibitor (serpin B1), Plasma protease C1 inhibitor, Plasminogen activator inhibitor, Serpin B9/maspin, Serpin E3, and Serpin H1.

In some embodiments, the present invention relates to a method for determining whether a subject having a low grade dysplasia is at risk of having a cancer comprising determining the level of at least one immune marker in a biological sample obtained from the subject wherein the immune marker is T cells CD4 naive and wherein the expression level of the immune marker correlates with the risk of having cancer.

More particularly, the present invention relates to a method for determining whether a subject having a low grade bronchial dysplasia is at risk of having a lung cancer comprising determining the level of at least one immune marker in a biological sample obtained from the subject wherein the immune marker is T cells CD4 naive and wherein the expression level of the immune marker correlates with the risk of having cancer.

As used herein, the term “T cell” has its general meaning in the art and refers to a type of lymphocytes that play an important role in cell-mediated immunity and are distinguished from other lymphocytes, such as B cells, by the presence of a T-cell receptor (TCR) on the cell surface. In particular, T cells are characterised by the expression of CD3. The term “CD3” refers to the protein complex associated with the T cell receptor is composed of four distinct chains. In mammals, the complex contains a CD3γ chain, a CD3δ chain, and two CD3ε chains. These chains associate with the TCR and the ζ-chain (zeta-chain) to generate an activation signal in T lymphocytes. The TCR, ζ-chain, and CD3 molecules together constitute the TCR complex.

As used herein, the term “CD4” has its general meaning in the art and refers to the T-cell surface glycoprotein CD4. CD4 is a co-receptor of the T cell receptor (TCR) and assists the latter in communicating with antigen-presenting cells. The TCR complex and CD4 each bind to distinct regions of the antigen-presenting MHCII molecule—α1/β1 and β2, respectively.

As used herein, the term “CD4+ T cells” has its general meaning in the art and refers to a subset of T cells which express CD4 on their surface. CD4+ T cells are T helper cells, which either orchestrate the activation of macrophages and CD8+ T cells (Th-1 cells), the production of antibodies by B cells (Th-2 cells) or which have been thought to play an essential role in autoimmune diseases (Th-17 cells).

A “naive T cell” is a T cell that has differentiated in bone marrow, and successfully undergone the positive and negative processes of central selection in the thymus. Naive T cells are commonly characterized by the surface expression of L-selectin (CD62L) and C-C Chemokine receptor type 7 (CCR7); the absence of the activation markers CD25, CD44 or CD69; and the absence of memory CD45RO isoform. They also express functional IL-7 receptors, consisting of subunits IL-7 receptor-α, CD127, and common-γ chain, CD132.

In some embodiments, the present invention relates to a method for determining whether a subject having a low grade dysplasia is at risk of having a cancer comprising determining the level of at least one immune marker in a biological sample obtained from the subject wherein the immune marker is selected from the group consisting of TNFRSF18 (GITR), IL18, TNFRSF14 (HVEM), TNFSF4, and TNFRSF17 (BCMA) and wherein the expression level of the immune marker correlates with the risk of having cancer.

In some embodiments, the present invention relates to a method for determining whether a subject having a low grade bronchial dysplasia is at risk of having a lung cancer comprising determining the level of at least one immune marker in a biological sample obtained from the subject wherein the immune marker is selected from the group consisting of TNFRSF18 (GITR), IL18, TNFRSF14 (HVEM), TNFSF4, and TNFRSF17 (BCMA) and wherein the expression level of the immune marker correlates with the risk of having cancer.

As used herein, the term “TNFRSF18” or “GITR” has its general meaning in the art and refers to the tumor necrosis factor receptor superfamily member 18 also known as glucocorticoid-induced TNFR-related protein. This receptor has been shown to have increased expression upon T-cell activation, and it is thought to play a key role in dominant immunological self-tolerance maintained by CD25+/CD4+ regulatory T cells.

As used herein, the term “IL18” has its general meaning in the art and refers to the unterleukin—18, also known as interferon-gamma inducing factor. IL18 is a protein which in humans is encoded by the IL18 gene. IL-18 works by binding to the interleukin-18 receptor, and together with IL-12, it induces cell-mediated immunity following infection with microbial products like lipopolysaccharide (LPS).

As used herein, the term “TNFRSF14” or “HVEM” has its general meaning in the art and refers to the tumor necrosis factor receptor superfamily member 14 also known as herpesvirus entry mediator (HVEM). TNFRSF14 is a human cell surface receptor of the TNF-receptor superfamily. The protein functions in signal transduction pathways that activate inflammatory and inhibitory T-cell immune response. It binds herpes simplex virus (HSV) viral envelope glycoprotein D (gD), mediating its entry into cells.

As used herein, the term “TNFSF4” has its general meaning in the art and refers to the tumor necrosis factor ligand superfamily member 4. The term is also known as OX40L or CD52. TNFSF4 is a cytokine that binds to TNFRSF4 and co-stimulates T-cell proliferation and cytokine production.

As used herein, the term “TNFRSF17” or “BCMA” has its general meaning in the art and refers to tumor necrosis factor receptor superfamily member 17 also known as B-cell maturation antigen. TNFRSF17 is a cell surface receptor of the TNF receptor superfamily which recognizes B-cell activating factor (BAFF). This receptor is preferentially expressed in mature B lymphocytes, and may be important for B cell development.

Methods for Determining Whether a Subject Having a High Grade Dysplasia is at Risk of Having a Cancer:

In some embodiments, the present invention relates to a method for determining whether a subject having a high grade dysplasia is at risk of having a cancer comprising determining the level of at least one immune marker in a biological sample obtained from the subject wherein the immune marker is selected from the group consisting of co-inhibitory molecules, co-stimulatory molecules, immunosuppressive interleukins and immunostimulatory interleukins and wherein the expression level of the immune marker correlates with the risk of having cancer.

More particularly, the present invention relates to a method for determining whether a subject having a high grade bronchial dysplasia is at risk of having a lung cancer comprising determining the level of at least one immune marker in a biological sample obtained from the subject wherein the immune marker is selected from the group consisting of co-inhibitory molecules, co-stimulatory molecules, immunosuppressive interleukins and immunostimulatory interleukins and wherein the expression level of the immune marker correlates with the risk of having cancer.

As used herein, the term “co-stimulatory molecule” has its general meaning in the art and refers to a group of immune cell surface receptor in T cell whose engagement by specific ligand appears to be necessary for a complete activation response following antigen receptor binding by antigen. In some embodiments, the co-stimulatory molecule is selected from the group consisting of CD137, GITR, ICOS, TNFRSF25 and CD86.

As used herein, the term “co-inhibitory molecule” has its general meaning in the art and refers to a group of immune cell surface receptor in T cell whose engagement by specific ligand thereby slowing down or preventing activation response following antigen receptor binding by antigen. In some embodiments, the co-inhibitory molecule is selected from the group consisting of PDL1, PD1, IDO1, CTLA4, and TIGIT.

As used herein, the term “immunostimulatory interleukin” has its general meaning in the art and refers to an interleukin that induces the activity of the immune system. Immunostimulatory interleukins act by enhancing the function of responding immune cells (including, for example, T cells) directly (e.g., by acting on the immune cell) or indirectly (by acting on other mediating cells). In some embodiments, the immunostimulatory interleukin is selected from the group consisting of IL-18 and IFNG.

As used herein, the term “immunosuppressive interleukin” has its general meaning in the art and refers to an interleukin that inhibits, slows or reverses the activity of the immune system. Immunosuppressive interleukins act by suppressing the function of responding immune cells (including, for example, T cells) directly (e.g., by acting on the immune cell) or indirectly (by acting on other mediating cells). In some embodiments, the immunosuppressive interleukin is selected from the group consisting of IL6, IL10, and TGFβ.

Methods for Quantifying the Immune Markers:

In some embodiments, the level of the immune marker is determined by immunohistochemistry (IHC). Immunohistochemistry typically includes the following steps i) fixing said tissue sample with formalin, ii) embedding said tissue sample in paraffin, iii) cutting said tissue sample into sections for staining, iv) incubating said sections with the binding partner specific for the immune marker, v) rinsing said sections, vi) incubating said section with a biotinylated secondary antibody and vii) revealing the antigen-antibody complex with avidin-biotin-peroxidase complex. Accordingly, the tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the immune marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 125I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the immune marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the immune marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms (e.g., Aperio Spectrum Software, Automated QUantitatative Analysis platform (AQUA® platform), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the immune marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. In some embodiments, a score is given to the sample that is a numerical representation of the intensity or amount of the histochemical staining of the sample, and represents the amount of target biomarker (e.g., the immune marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the immune marker (e.g. an antibody as above descried), ii) proceeding to digitalisation of the slides of step a. by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity of stained cells in each unit whereby the number or the density of cells stained of each unit is assessed.

Multiplex tissue analysis techniques are particularly useful for quantifying several markers in the tissue sample. Such techniques should permit at least five, or at least ten or more biomarkers to be measured from a single tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.).

In some embodiments, the L-IHC method can be performed on any of a variety of tissue samples, whether fresh or preserved. The samples included core needle biopsies that were routinely fixed in 10% normal buffered formalin and processed in the pathology department. Standard five μη thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10μη thick coated polymer backbone with 0.4μη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.). That technique is described in U.S. Pat. No. 7,473,532.

In some embodiments, the method of the present invention utilized the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the subject, ii) TMA samples are then stained with anti-antibodies having specificity of the protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the level of the immune marker is determined at nucleic acid level. Typically, the level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Puhlication No. 2003/0165951 as well as PCT Puhlication No. 99/26299 (puhlished May 27, 1999). Separate populations of semiconductor nanocrystals can he produced that are identifiable based on their different spectral characteristics. For example, semiconductor nanocrystals can he produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as ³H), metal chelates such as DOTA and DPTA chelates of radioactive or paramagnetic metal ions like Gd3+, and liposomes. Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase. Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Puhlication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will he appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR.

In some embodiments, the level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tissue sample with a probe library, such that the presence of the target in the sample creates a probe pair—target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe,electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Level of a gene may be expressed as absolute level or normalized level. Typically, levels are normalized by correcting the absolute level of a gene by comparing its expression to the expression of a gene that is not a relevant for determining the risk. This normalization allows the comparison of the level in one sample, e.g., a subject sample, to another sample, or between samples from different sources.

In some embodiments, when the sample is a bodily fluid sample, the level of the immune marker is determined by an immunoassay. Such assays include, for example, competition assays, direct reaction assays sandwich-type assays and immunoassays (e.g. ELISA). The assays may be quantitative or qualitative. There are a number of different conventional assays for detecting formation of an immunocomplex. For example, the detecting step can comprise performing an ELISA assay, performing a lateral flow immunoassay, performing an agglutination assay, analyzing the sample in an analytical rotor, or analyzing the sample with an electrochemical, optical, or opto-electronic sensor. These different assays are well-known to those skilled in the art. In some embodiments, the devices are useful for performing an immunoassay according to the present invention. For example, in some embodiments, the device is a lateral flow immunoassay device. In some embodiments, the device is an analytical rotor. In some embodiments, the device is a dot blot. In some embodiments, the device is a tube or a well, e.g., in a plate suitable for an ELISA assay. In some embodiments, the device is an electrochemical sensor, an optical sensor, or an opto-electronic sensor. The presence and amount of the immunocomplex may be detected by methods known in the art, including label-based and label-free detection. For example, label-based detection methods include addition of a secondary antibody that is coupled to an indicator reagent comprising a signal generating compound. The secondary antibody may be an anti-human IgG antibody. Indicator reagents include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors and magnetic particles. Examples of enzyme conjugates include alkaline phosphatase, horseradish peroxidase and beta-galactosidase. Methods of label-free detection include surface plasmon resonance, carbon nanotubes and nanowires, and interferometry. Label-based and label-free detection methods are known in the art and disclosed, for example, by Hall et al. (2007) and by Ray et al. (2010) Proteomics 10:731-748. Detection may be accomplished by scanning methods known in the art and appropriate for the label used, and associated analytical software. In some embodiments, fluorescence labeling and detection methods are used to detect the immunocomplexes. A particularly useful assay format is a lateral flow immunoassay format. Antibodies to human or animal (e.g., dog, mouse, deer, etc.) immunoglobulins, or staph A or G protein antibodies, can be labeled with a signal generator or reporter (e.g., colloidal gold) that is dried and placed on a glass fiber pad (sample application pad or conjugate pad). Another assay is an enzyme linked immunosorbent assay, i.e., an ELISA. Typically in an ELISA, the immune markers are adsorbed to the surface of a microtiter well directly or through a capture matrix (e.g., an antibody). Residual, non-specific protein-binding sites on the surface are then blocked with an appropriate agent, such as bovine serum albumin (BSA), heat-inactivated normal goat serum (NGS), or BLOTTO (a buffered solution of nonfat dry milk which also contains a preservative, salts, and an antifoaming agent). The well is then incubated with the sample. The sample can be applied neat, or more often it can be diluted, usually in a buffered solution which contains a small amount (0.1-5.0% by weight) of protein, such as BSA, NGS, or BLOTTO. After incubating for a sufficient length of time to allow specific binding to occur, the well is washed to remove unbound protein and then incubated with an optimal concentration of an appropriate anti-immunoglobulin antibody (e.g., for human subjects, an anti-human immunoglobulin (αHulg) from another animal, such as dog, mouse, cow, etc. that is conjugated to an enzyme or other label by standard procedures and is dissolved in blocking buffer. The label can be chosen from a variety of enzymes, including horseradish peroxidase (HRP), beta-galactosidase, alkaline phosphatase, glucose oxidase, etc. Sufficient time is allowed for specific binding to occur again, then the well is washed again to remove unbound conjugate, and a suitable substrate for the enzyme is added. Color is allowed to develop and the optical density of the contents of the well is determined visually or instrumentally (measured at an appropriate wave length).

In some embodiments, when multi-quantification is required, use of beads bearing binding partners of interest may be preferred. In some embodiments, the bead may be a cytometric bead for use in flow cytometry. Such beads may for example correspond to BD™ Cytometric Beads commercialized by BD Biosciences (San Jose, Calif.). Typically cytometric beads may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

Predetermined Reference Values:

In some embodiments, the method of the present invention further comprises comparing the expression level of the immune marker with a predetermined reference value wherein detecting a difference between the expression level of the immune marker and the predetermined reference value indicates whether the subject is or is not at risk of having cancer.

In some embodiments, the predetermined reference value is a relative to a number or value derived from population studies, including without limitation, subjects of the same or similar age range, subjects in the same or similar ethnic group, and subjects having the same severity of premalignant lesion. Such predetermined reference values can be derived from statistical analyses and/or risk prediction data of populations obtained from mathematical algorithms and computed indices. In some embodiments, retrospective measurement of the level of the immune marker in properly banked historical subject samples may be used in establishing these predetermined reference values. Accordingly, in some embodiments, the predetermined reference value is a threshold value or a cut-off value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the immune marker in a group of reference, one can use algorithmic analysis for the statistic treatment of the measured levels of the immune marker in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

Typically, an increase in the level of CD58, SERPIN members, T cell CD4 naive, TNFRSF18 (GITR), and IL18 in low grade dysplasia compared to a standard level observed in a control population (e.g. a population of subjects having premalignant lesions that never progress to cancer) is associated with an increased risk of having a cancer.

Typically, a decrease in the level of TNFRSF14 (HVEM), TNFSF4, and TNFRSF17 (BCMA) in low grade dysplasia compared to a standard level observed in a control population (e.g. a population of subjects having premalignant lesions that never progress to cancer) is associated with an increased risk of having a cancer.

Typically, an increase in the level of co-inhibitory molecules, co-stimulatory molecules, immunosuppressive interleukins and immunostimulatory interleukins in high grade dysplasia compared to a standard level observed in a control population (e.g. a population of subjects having premalignant lesions that never progress to cancer) is associated with an increased risk of having a cancer.

Methods for Implementing a Score:

In some embodiments, a score which is a composite of the expression levels of the different immune markers is determined and compared to the predetermined reference value wherein a difference between said score and said predetermined reference value is indicative whether the subject is at risk of having cancer.

In some embodiments, the method of the invention comprises the use of a classification algorithm typically selected from Linear Discriminant Analysis (LDA), Topological Data Analysis (TDA), Neural Networks, Support Vector Machine (SVM) algorithm and Random Forests algorithm (RF) such as described in the Example. In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

In some embodiments, the method of the present invention comprises a) quantifying the level of a plurality of immune markers in the sample; b) implementing an algorithm on data comprising the quantified plurality of immune markers so as to obtain an algorithm output; c) determining the probability that the subject will develop a cancer from the algorithm output of step b).

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

Methods for the Prophylactic Treatment of Cancer in a Subject Having at Least One Premalignant Lesion:

A further object of the present invention relates to a method for the prophylactic treatment of cancer in a subject having at least one premalignant lesion comprising administering to the subject a therapeutically effective amount of at least one chemopreventive agent.

As used herein, the terms “prophylaxis” or “prophylactic use” and “prophylactic treatment” as used herein, refer to any medical or public health procedure whose purpose is to prevent a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a subject with the disease.

In some embodiments, the subject has been considered as being at risk of having cancer by the predictive method of the present invention.

In some embodiments, the chemopreventive agent is selected from the group consisting of alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g.; calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the chemopreventive agent is an immune checkpoint inhibitor. As used herein, the term “immune checkpoint inhibitor” has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, GITR antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab. Other molecules with similar mechanisms that would be developed in the future are also potential candidate for cancer chemoprevention.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab. Other molecules with similar mechanisms that would be developed in the future are also potential candidate for cancer chemoprevention.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057. Other molecules with similar mechanisms that would be developed in the future are also potential candidate for cancer chemoprevention.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, 3-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof. Other molecules with similar mechanisms that would be developed in the future are also potential candidate for cancer chemoprevention.

In some embodiments, the chemopreventive agent is an inhibitor of an immunosuppressive cytokine.

As used herein, the expression “inhibitor of an immunosuppressive cytokine” refers to a molecule that partially or fully blocks, inhibits, or neutralizes a biological activity or expression of an immunosuppressive cytokine. Thus the inhibitor can be a molecule of any type that interferes with the signaling associated with at least immunosuppressive cytokine in a cell, for example, either by decreasing transcription or translation of cytokine-encoding nucleic acid, or by inhibiting or blocking cytokine polypeptide activity, or both. Examples of inhibitors include, but are not limited to, antisense polynucleotides, interfering RNAs, catalytic RNAs, RNA-DNA chimeras, cytokine-specific aptamers, anti-cytokine antibodies, cytokine-binding fragments of anti-cytokine antibodies, cytokine-binding small molecules, cytokine-binding peptides, and other polypeptides that specifically bind to the cytokine such that the interaction between the inhibitor and the targeted cytokine results in a reduction or cessation of the cytokine activity or expression. In some embodiments, the inhibitor inhibits the interaction between the immunosuppressive cytokine and one of its receptor. Thus additional examples of inhibitors include receptor-specific aptamers, anti-receptor antibodies, receptor-binding fragments of anti-receptor antibodies, receptor-binding small molecules, receptor-binding peptides, and other polypeptides that specifically bind to the cytokine receptor such that the interaction between the inhibitor and the receptor results in a reduction or cessation of the cytokine activity, In some embodiments, the inhibitor is selected from the group consisting of IL6 inhibitors, IL10 inhibitors and TGFβ inhibitors.

In some embodiments, the inhibitor of IL6, IL10 or TGFβ is an antibody. As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

In some embodiments, the antibody is specific for the cytokine. In some embodiments, the antibody is specific for one receptor of the cytokine.

Antibodies showing TGFβ inhibitory activities are part of the common general knowledge. For example, monoclonal and polyclonal antibodies directed against one or more isoforms of TGFβ have been described in U.S. Pat. No. 5,571,714; WO 97/13844; and WO 00/66631; WO 05/097832; WO 05/101149; WO 06/086469. Antibodies directed against TGFβ receptors have also bee described in Flavell et al., Nat. Rev. Immunol. 2:46-53 (2002; U.S. Pat. Nos. 5,693,607; 6,001,969; 6,008,011; 6,010,872; WO 92/00330; WO 93/09228; WO 95/10610; and WO 98/48024.

Non-limiting examples of anti-IL-6 antibodies or IL-6 binding fragment thereof include Siltuximab, Olokizumab, ALD518 (BMS-945429), C326, Sirukumab, Elsilimomab and Clazakizumab.

Patents and patent publications related to anti-IL-6R antibodies include. U.S. Pat. No. 5,171,840 (Kishimoto), U.S. Pat. No. 5,480,796 (Kishimoto), U.S. Pat. No. 5,670,373 (Kishimoto), U.S. Pat. No. 5,851,793 (Kishimoto), U.S. Pat. No. 5,990,282 (Kishimoto), U.S. Pat. No. 6,410,691 (Kishimoto), U.S. Pat. No. 6,428,979 (Kishimoto), U.S. Pat. No. 5,795,965 (Tsuchiya et al.), U.S. Pat. No. 5,817,790 (Tsuchiya et al.), U.S. Pat. No. 7,479,543 (Tsuchiya et al.), US 2005/0142635 (Tsuchiya et al.), U.S. Pat. No. 5,888,510 (Kishimoto et al.), US 2001/0001663 (Kishimoto et al.), US 2007/0036785 (Kishimoto et al.), U.S. Pat. No. 6,086,874 (Yoshida et al.), U.S. Pat. No. 6,261,560 (Tsujinaka et al.), U.S. Pat. No. 6,692,742 (Nakamura et al.), U.S. Pat. No. 7,566,453 (Nakamura et al.), U.S. Pat. No. 7,771,723 (Nakamura et al.), US 2002/0131967 (Nakamura et al.), US 2004/0247621 (Nakamura et al.), US 2002/0187150 (Mihara et al.), US 2005/0238644 (Mihara et al.), US 2009/0022719 (Mihara et al.), US 2006/0134113 (Mihara), U.S. Pat. No. 6,723,319 (Ito et al.), U.S. Pat. No. 7,824,674 (Ito et al.), US 2004/0071706 (Ito et al.), U.S. Pat. No. 6,537,782 (Shibuya et al.), U.S. Pat. No. 6,962,812 (Shibuya et al.), WO 00/10607 (Akihiro et al.), US 2003/0190316 (Kakuta et al.), US 2003/0096372 (Shibuya et al.), U.S. Pat. No. 7,320,792 (Ito et al.), US 2008/0124325 (Ito et al.), US 2004/0028681 (Ito et al.), US 2008/0124325 (Ito et al.), US 2006/0292147 (Yoshizaki et al.), US 2007/0243189 (Yoshizaki et al.), US 2004/0115197 (Yoshizaki et al.), US 2007/0148169 (Yoshizaki et al.), U.S. Pat. No. 7,332,289 (Takeda et al.), U.S. Pat. No. 7,927,815 (Takeda et al.), U.S. Pat. No. 7,955,598 (Yoshizaki et al.), US 2004/0138424 (Takeda et al.), US 2008/0255342 (Takeda et al.), US 2005/0118163 (Mizushima et al.), US 2005/0214278 (Kakuta et al.), US 2008/0306247 (Mizushima et al.), US 2009/0131639 (Kakuta et al.), US 2006/0142549 (Takeda et al.), U.S. Pat. No. 7,521,052 (Okuda et al.), US 2009/0181029 (Okuda et al.), US 2006/0251653 (Okuda et al.), US 2009/0181029 (Okuda et al.), US 2007/0134242 (Nishimoto et al.), US 2008/0274106 (Nishimoto et al.), US 2007/0098714 (Nishimoto et al.), US 2010/0247523 (Kano et al.), US 2006/0165696 (Okano et al.), US 2008/0124761 (Goto et al.), US 2009/0220499 (Yasunami), US 2009/0220500 (Kobara), US 2009/0263384 (Okada et al.), US 2009/0291076 (Morichika et al.), US 2009/0269335 (Nakashima et al.), US 2010/0034811 (Ishida), US 2010/0008907 (Nishimoto et al.), US 2010/0061986 (Takahashi et al.), US 2010/0129355 (Ohguro et al.), US 2010/0255007 (Mihara et al.), US 2010/0304400 (Stubenrach et al.), US 2010/0285011 (Imaeda et al.), US 2011/0150869 (Mitsunaga et al.), WO 2011/013786 (Maeda) and US 2011/0117087 (Franze et al.).

In some embodiments, the anti-IL6R antibody is Tocilizumab.

In some embodiments, the IL-6, IL-10 or TGFβ inhibitor is a small organic molecule.

In some embodiments, examples of small organic molecules that can be used as TGFβ inhibitors include but are not limited to those described in WO 02/062753; WO 02/062776; WO 02/062787; WO 02/062793; WO 02/062794; WO 02/066462; WO 02/094833; WO 03/087304; WO 03/097615; WO 03/097639; WO 04/010929; WO 04/021989; WO 04/022054; WO 04/024159; WO 04/026302; WO 04/026871; U.S. Pat. No. 6,184,226; WO 04/016606; WO 04/047818; WO 04/048381; WO 04/048382; WO 04/048930; WO 04/050659; WO 04/056352; WO 04/072033; WO 04/087056 WO 05/010049; WO 05/0032481; WO 05/0065691; WO 05/092894; WO 06/026305; WO 06/026306; and WO 06/052568. In some embodiments, the TGF-β inhibitor is selected from, but not limited to the group consisting of SB431542 (4-[4-(1,3-benzodioxol-5-yl)-5-(2-pyridinyl)-1H-imidazol-2-yl]benzamide), SB525334 (6-[2-(1,1-Dimethylethyl)-5-(6-methyl-2-pyridinyl)-1H-imidazol-4-yl]quinoxaline), Ki26894 (Kirin Brewery Company, Gunma, Japan, (Ehata et al Cancer Sci 98): 127-133), LY364947 (4-[3-(2-Pyridinyl)-1H-pyrazol-4-yl]-quinoline), SD-208 (2-(5-Chloro-2-fluorophenyl)-4-[(4-pyridyl)amino]pteridine), SD-093 (2-(2-fluorophenyl)-N-pyridin-4-ylpyrido[2,3-d]pyrimidi-4-amine) (U.S. Pat. No. 6,476,031), SM16 (4-(5-(benzo[d][1,3]dioxol-5-yl)-4-(6-methylpyridin-2-yl)-1H-imidazol-2-yl)bicyclo[2.2.2]octane-1-carboxamide), Ly2109761(4-[2-[4-(2-pyridin-2-yl-5,6-dihydro-4H-pyrrolo[1,2-b]pyrazol-3-yl)quinolin-7-yl]oxyethyl]morpholine), Ly2157299 (2-(6-methyl-pyridin-2-yl)-3-[6-amido-quinolin-4-yl)-5,6-dihydro-4H-pyrrolo[1,2-b]pyrazole monohydrate), K02288(3-[6-amino-5-(3,4,5-trimethoxy-phenyl)-pyridin-3-yl]-plienol), SB505124 (2-[4-(1,3-Benzodioxol-5-yl)-2-(1,1-dimethylethyl)-1H-imidazol-5-yl]-6-methyl-pyridine), LDN-193189 (4-(6-(4-(piperazin-1-yl) phenyl) pyrazolo[1,5-a]pyrimidin-3-yl)quinoline hydrochloride), GW788388 (4-[4-[3-(2-Pyridinyl)-1H-pyrazol-4-yl]-2-pyridinyl]-N-(tetrahydro-2H-pyran-4-yl)-benzamide), Ly580276 (3-(4-fluorophenyl)-2-(6-methylpyridin-2-yl)-5,6-dihydro-4H-pyrrolo[1,2-b]pyrazole), EW-7203 (3-((5-([1,2,4]triazolo[1,5-a]pyridin-6-yl)-4-(6-methylpyridin-2-yl)thiazol-2-ylamino)methyl)benzonitrile), EW-7195 (3-[methyl-[5-(6-methylpyridin-2-yl)-4-([1,2,4]triazolo[1,5-a]pyridin-6-yl) H-imidazol-2-yl]amino]benzonitrile), EW-7197 (N-[[4-([1,2,4]triazolo[1,5-a]pyridin-6-yl)-5-(6-methylpyridin-2-yl)-1H-imidazol2-yl]methyl]-2-fluoroaniline), YR-290 (N-phenylacetyl-1,3,4,9-tetrahydro-1H-beta-carboline), A 83-01(3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1H-imidazol-2-yl]benzamide), RepSox[alternatively E-616452, SJN 2511] (2-(3-(6-Methylpyridine-2-yl)-1H-pyrazol-4-yl)-1,5-naphthyridine), R268712 (4-[2-Fluoro-5-[3-(6-methyl-2-pyridinyl)-1H-pyrazol-4-yl]phenyl]-1H-pyrazole-1-ethanol) (or mixtures or combinations thereof, or and pharmaceutically acceptable salts thereof.

In some embodiments, the IL-6 inhibitor or IL-10 inhibitor is selected from JAK inhibitors. As used herein the term “JAK” has its general meaning in the art and refers to the family of Janus kinases (JAKs) which are cytoplasmic tyrosine kinases that transduce cytokine (e.g. IL-6 or IL-10) signaling from membrane receptors to STAT transcription factors. Four JAK family members are described, JAK1, JAK2, JAK3 and TYK2 and the term JAK may refer to all the JAK family members collectively or one or more of the JAK family members as the context indicates. As used herein the term “JAK inhibitor” is intended to mean compounds inhibit the activity or expression of at least JAK2. JAK inhibitors down-regulate the quantity or activity of JAK molecules. One activity of JAK2 is to phosphorylate a STAT protein. Therefore an example of an effect of a JAK inhibitor is to decrease the phosphorylation of one or more STAT proteins. The inhibitor may inhibit the phosphorylated form of JAK2 or the non-phosphorylated form of JAK2. In some embodiments, the JAK inhibitor is a selective JAK2 inhibitor. By “selective” is meant that the compound binds to or inhibits JAK2 with greater affinity or potency, respectively, compared to at least one other JAK (e.g., JAK1, JAK3 and/or TYK2). Selectivity can be at least about 5-fold, at least about 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the Km of each enzyme. JAK inhibitors are well known in the art. For example, JAK inhibitors include phenylaminopyrimidine compounds (WO2009/029998), substituted tricyclic heteroaryl compounds (WO2008/079965), cyclopentyl-propanenitrile compounds (WO2008/157208 and WO2008/157207), indazole derivative compounds (WO2008/114812), substituted ammo-thiophene carboxylic acid amide compounds (WO2008/156726), naphthyridine derivative compounds (WO2008/112217), quinoxaline derivative compounds (WO2008/148867), pyrrolopyrimidine derivative compounds (WO2008/119792), purinone and imidazopyridinone derivative compounds (WO2008/060301), 2,4-pyrimidinediamine derivative compounds (WO2008/118823), deazapurine compounds (WO2007/117494) and tricyclic heteroaryl compounds (WO2008/079521). Examples of JAK inhibitors include compounds disclosed in the following publications: US2004/176601, US2004/038992, US2007/135466, US2004/102455, WO2009/054941, US2007/134259, US2004/265963, US2008/194603, US2007/207995, US2008/260754, US2006/063756, US2008/261973, US2007/142402, US2005/159385, US2006/293361, US2004/205835, WO2008/148867, US2008/207613, US2008/279867, US2004/09799, US2002/055514, US2003/236244, US2004/097504, US2004/147507, US2004/176271, US2006/217379, US2008/092199, US2007/043063, US2008/021013, US2004/152625, WO2008/079521, US2009/186815, US2007/203142, WO2008/144011, US2006/270694 and US2001/044442. JAK inhibitors further include compounds disclosed in the following publications: WO2003/011285, WO2007/145957, WO2008/156726, WO2009/035575, WO2009/054941, and WO2009/075830. JAK inhibitors further include compounds disclosed in the following patent applications: U.S. Ser. Nos. 61/137,475 and 61/134,338. Specific JAK inhibitors include AG490, AUB-6-96, AZ960, AZD1480, baricitinib (LY3009104, INCB28050), BMS-911543, CEP-701, CMP6, CP352,664, CP690,550, CYT-387, INCB20, Jak2-IA, lestaurtinib (CEP-701), LS104, LY2784544, NS018, pacritinib (SB1518), Pyridone 6, ruxolitinib (INCB018424), SB1518, TG101209, TG101348 (SAR302503), TG101348, tofacitinib (CP-690,550), WHI-PI 54, WP1066, XL019, and XLOI 9. Ruxolitinib (Jakafi™, INCB018424; (3R)-3-cyclopentyl-3-[4-(7H-pyrrolo[2,3-d]pyrimidin-4-yl)pyrazol-1-yl]propanenitrile) is a potent, orally available, selective inhibitor of both JAK1 and JAK2 of the JAK-STAT signaling pathway. CYT387 is an inhibitor of Janus kinases JAK1 and JAK2, acting as an ATP competitor with IC50 values of 11 and 18 nM, respectively. TG101348 (SAR302503) is an orally available inhibitor of Janus kinase 2 (JAK-2). TG101348 acts as a competitive inhibitor of protein kinase JAK-2 with IC50=6 nM; related kinases FLT3 and RET are also sensitive, with IC50=25 nM and IC50=17 nM, respectively. AZD1480 is an orally bioavailable inhibitor of Janus-associated kinase 2 (JAK2) with potential antineoplastic activity. JAK2 inhibitor AZD 1480 inhibits JAK2 activation, leading to the inhibition of the JAK/STAT (signal transducer and activator of transcription) signaling including activation of STAT3. Lestaurtinib (CEP-701) is a tyrosine kinase inhibitor structurally related to staurosporine. Pacritinib (SB 1815) is an orally bioavailable inhibitor of JAK2 and the JAK2 mutant JAK2V617F. Pacritinib competes with JAK2 for ATP binding, which may result in inhibition of JAK2 activation, inhibition of the JAK-STAT signaling pathway, and therefore caspase-dependent apoptosis. Baricitinib (LY3009104, INCB28050) is an orally bioavailable inhibitor of JAK1 and JAK2 with IC50=5.9 nm and IC50=5.7, nm respectively. Baricitinib preferentially inhibits JAK1 and JAK2, with 10-fold selectivity over Tyk2 and 100-fold over JAK3. XL019 is an orally bioavailable inhibitor of Janus-associated kinase 2 (JAK2). XL019 inhibits the activation of JAK2 as well as the mutated form JAK2V617F. NS018 is a potent JAK2 inhibitor with some inhibition of Src-family kinases. NS018 has been shown to be highly active against JAK2 with a 50% inhibition (IC50) of <1 nM, and had 30-50-fold greater selectivity for JAK2 over other JAK-family kinases.

In some embodiments, IL-6 inhibitors include peptides that block IL-6 signaling such as those described in any of U.S. Pat. Nos. 6,599,875; 6,172,042; 6,838,433; 6,841,533; and 5,210,075. Also, IL-6 inhibitors according to the invention may include p38 MAP kinase inhibitors such as those reported in US20070010529, given the role of p38 MAP kinase in production of cytokines such as IL-6. Further, IL-6 inhibitors according to the invention include the glycoalkaloid compounds reported in US20050090453 as well as other IL-6 antagonist compounds isolatable using the screening assays reported therein.

In some embodiments, the inhibitor is an inhibitor of IL6, IL10 or TGFβ expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of the cytokine mRNA by binding thereto and thus preventing protein translation or increasing mRNA degradation, thus decreasing the level of the cytokine, and thus activity, in a cell. For example, antisense oligonucleotides of at least about 15 bases and complementary to unique regions of the mRNA transcript sequence encoding the cytokine can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. the cytokine gene expression can be reduced by contacting a patient or cell with a small double stranded RNA (dsRNA), or a vector or construct causing the production of a small double stranded RNA, such that the cytokine gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing the cytokine. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. In some embodiments, the inhibitor of expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences.

The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the inhibitor is selected from the group consisting of IL-6 soluble receptors, IL-10 soluble receptors, TGFβ soluble receptors.

In some embodiments, the chemopreventive agents are immunomodulatory antigen such as a vaccine against an immune checkpoint inhibitor or a suppressive cytokine or suppressive protein. Preferred immune checkpoint inhibitors are vaccine against these molecules that specifically generate an adaptive immune response (T-cell response and B-cell response) inducing or expanding T-cells and B-cells having specificities against these immune checkpoint inhibitor or suppressive cytokine or suppressive protein. Examples of vaccine against immune checkpoint inhibitor includes proteins or peptides of PD-1, PD-L1, PD-L2 CTLA-4, VISTA, TIM-3, LAG-3, GITR, IDO, KIR2D, A2AR, B7-H3, B7-H4, and BTLA. In some embodiments, the inhibitor is vaccine against suppressive cytokine or suppressive molecules such as IL6, IL10 and TGFβ.

In some embodiments, the chemopreventive agent is administered locally in the premalignant lesion or by systemic approaches to the subject. When possible, the agent is administered via a local route. Typically, when the subject suffers from a premalignant lesion of the skin, the chemopreventive agent is topically administered to the subject. Eventhough systemic route is more at risk of sides effects including auto-immune responses, it is required in many cases of sites that are not accessible by local route or in case of field of cancerization.

The invention will be further illustrated by the following figures and examples. However, these examples and figures should not be interpreted in any way as limiting the scope of the present invention.

### FIGURES

FIG. 1. Genes encoding form CD58 and SERPIN members had a biphasic increase in low-grade dysplasia.

FIG. 2. Continuous shift of immune status for CD4 T cells. Significant differences per stage were highlighted with black asterisks at FDR<0.1, or grey otherwise (Mann-Whitney test, *** p<0.001, ** 0.001≥p<0.01, * 0.01≥p<0.05, −0.05≥p<0.1, BH adjustment).

FIG. 3 Immune evasion before tumor invasion in early squamous lung carcinogenesis. A) Average expression of co-inhibitory molecules and immunosuppressive interleukins increases in high-grade and is further amplified in tumor samples (non-parametric, rank-based test, Dunn's pairwise multiple comparison test, *** p<0.001, ** 0.001≥p<0.01, * 0.01≥p<0.05, −0.05≥p<0.1). B) Positive fold changes in high-grade and SCC compared to their corresponding normal tissue expression were observed for co-stimulatory (e.g. CD137), co-inhibitory (e.g. TIGIT, PDL1), and suppressive interleukins (IL6, IL10). The p-values and coefficient estimates were derived from the linear mixed-effects model of the four molecular steps, adjusted for smoking history and previous cancer status as fixed effects, and patient as random effect.

### EXAMPLE

Methods:

1) Study Population

Bronchial biopsies were collected between 2003 and 2007 at the Jules Bordet Institute, Brussels, Belgium, during fluorescence bronchoscopy in current or former smokers with a smoking exposure of ≥30 pack-years. Former smokers were defined as individuals who had quit smoking for more than 6 months. The study was approved by the ethics committee of the Jules Bordet Institute and the patients gave informed consent. Based on the fact that high-grade lesions were rare and based on Dobbin et al.'s report³¹, we included at least twelve biopsies from each histological stage. The histopathological classification was performed by one pathologist (AH) on three independent blinded occasions. Any discordant diagnoses between successive evaluations were re-evaluated by the local team of pathologists using a multi-head microscope to obtain a consensus. Biopsies were classified using the 2004 histological WHO/IASLC classification of pre-invasive and invasive squamous lesions of the bronchus³². In addition, normal bronchial biopsies from 16 never-smokers were collected and pooled (same amount of RNA for each) for use as reference RNA.

A total of 122 biopsies from 77 individuals, 35 former and 42 current smokers, were studied. The median age was 62 years (range 42-78). The male/female ratio was 62/15. The 122 biopsies were distributed according to histology and fluorescence status as follows: 13 biopsies with normal histology and normofluorescent (8/5 biopsies from former/current smokers), 14 with normal histology and hypofluorescent (8/6), 15 hyperplasia (7/8), 15 metaplasia (5/10), 13 mild dysplasia (8/5), 13 moderate dysplasia (7/6), 12 severe dysplasia (2/10), 13 carcinoma in situ (CIS) (5/8) and 14 SCC (5/9). Among the 108 biopsies that were not SCC, 6 biopsies were taken in 4 patients having concurrent lung cancer. Among the 122 samples, matched FFPE blocks were found for 110 of them.

2) Sample Collection and RNA Extraction

During bronchoscopy, two biopsies were taken with clean forceps in the same area: one for routine histopathology and the second, immediately dropped in Tripure Isolation Reagent on ice, homogenized and frozen at −80° C. (Roche Diagnostics, Indianapolis, Ind., USA), for molecular studies. RNA extraction protocols have been previously described²⁷. Isolated RNAs were assessed for quantity and purity on the NanoDrop ND-1000 spectrophotometer (NanoDrop Technologies, Rockland, Del., USA) and for quality on the Agilent 2100 bioanalyser with RNA 6000 NanoAssay (Agilent Technologies, Palo Alto, Calif., USA). RNA was successfully extracted from 122 fresh frozen biopsies. The median yield of total RNA extracted from the biopsies was 1275 ng (range 244-11000 ng).

3) Acquisition and Analysis of Gene Expression Profiles

After amplification and labelling, cRNAs were hybridized on two Colours Whole Human Genome 4×44K arrays according to the recommendation of the provider (Agilent Technologies) (details in Text Si). Additional normalization steps were performed with Genespring GX, version 7.3.1, software (Agilent Technologies): 1) per spot (divide by control channel), 2) per chip (normalize to the median expression value across chip) and 3) per gene (normalize to median expression value across patients).

Several steps of data quality control were performed. Random principal component analysis showed that there were no outliers among the samples. The gene expression measurements in the cohort followed Gaussian distribution.

4) Identification of Linear Gene Expression Changes and Molecular Phenotypes

Monotonic gene expression alterations associated with developmental stages were identified using a linear model with mixed-effects. Each gene was modeled as a function of the developmental stage (factor variable), adjusting for smoking status, gender, and history of cancer as fixed effects. Because patient-level observations are not independent, we considered the parameter patient as a random effect. ANOVA tests compared the association of a gene and developmental stage to a null model. The false discovery rate (FDR) was calculated for each ANOVA³³ p-value using the method of Benjamini and Hochberg^{34,35}. Genes significantly associated with developmental stages was determined by an ANOVA FDR<0.001. Semi-supervised hierarchical clustering of these genes was then used to compare the nine different developmental stages.

5) Definition and Functional Characterization of Gene Modules

To identify trajectories of gene expression during development, we applied a WGCNA³⁶ on the genes significantly associated with developmental stages. WGCNA network construction and module detection was done using and signed network type, soft-thresholding power of 12, and a dendrogram cut height of 0.3 for merging modules. A minimum cluster size of 50 genes was used to define a module. A p-value ratio threshold of 0 was considered for reassigning genes across modules. The cluster eigengene (the first principal component of a cluster) value was used to evaluate the association of each module with the 9 stages of cancer. Thereby, we determined gene clusters (modules) of highly correlated genes with similar expression patterns across the nine developmental stages.

To functionally describe the gene modules, we used the cancer hallmark definitions from the mSigDB database³⁷ and applied the over-representation hypergeometric test using the R package clusterProfiler³⁸. In addition, we also used single-sample gene set enrichment analysis (ssGSEA)³⁹ on the full gene expression assay to determine whether the cancer hallmark genesets were enriched among the up-regulated or down-regulated genes within a sample (regardless of gene module). Probes were mapped to unique Entrez IDs. The genes were ranked by their z-score transformed expression values per sample. A minimum overlap of 5 genes with a given geneset was required. The enrichment score represents the degrees to which the genes from a given cancer hallmark geneset were up- or down-regulated within a sample.

6) Immune Cell-Type Signatures

To explore a large number of different immune cell subtypes and, even more, to examine their activation status, we compiled a large number of carefully annotated microarray gene expression profiles from almost 2000 publicly available experiments normalized with the frozen Robust Multi-array Averaging (fRMA) method. Building on our previous methods^{3,40,41}, we identified genes with specific expression for immune cell types, considering their naive, resting, and activated status (manuscript in preparation). Pan-cell type signatures were defined as genes expressed at similar levels in multiple cell types. For examples, the Myeloid-derived category comprised all subtypes of dendritic cells, eosinophils, monocytes, macrophages, neutrophils, and mast cells, while Macrophages-DC was a gene signature comprising common genes expressed in all studied subtypes of both macrophages and dendritic cells.

7) Immune Characterization from Gene-Expression Profiles

The defined immune signatures were used to explore a large variety immune cell types from the gene expression data at different histological stages of SCC development. First, we performed a hypergeometric test between the immune signatures and the gene modules, to pinpoint potential evolutionary trajectories of specific immune cell types.

We next applied the algorithm for absolute quantification implemented in CIBERSORT⁴² and deconvolved immune cell types expression from a mixed gene-expression signal according to the predefined LM22 signature.

Last, we performed single-sample GeneSet Enrichment Analysis (ssGSEA) using the in-house defined immune gene signatures. Thereby, for each immune cell type, we obtained an enrichment score per sample indicating the extent of up-regulation or down-regulation of the associated genes. The probe IDs were mapped to unique Entrez IDs. A minimum overlap of 5 genes was required.

8) Multiplex Immunohistochemistry and Multispectral Image Analysis

Matched formalin-fixed paraffin-embedded (FFPE) blocks of the 122 fresh frozen samples were available for 110 samples. Two four-μm tick slides were cut from the FFPE blocks, deparaffinized in clarene, rehydrated through an ethanol gradient and fixed in NBF (10% neutral buffered formalin). Slides were then stained according to the Opal 7-plex technology of PerkinElmer allowing the simultaneous visualization of 6 markers on the same slide. Therefore, at each of the 6 cycles of staining, antigen retrieval was performed via microwave treatment (MWT) in antigen retrieval solution pH6 or pH9 (AR6 or AR9) depending on the target, protein blocking was performed using Protein Block-Serum-free (Dako) for 15 min, and primary Abs were then incubated for 30 min at RT. Next, incubation with HRP Labelled Polymer mouse or rabbit (Dako EnVision+ System-HRP Labelled Polymer) was performed at room temperature for 15 min followed by TSA opal fluorophores (Opal 520, Opal 540, Opal 570, Opal 620, Opal 650 or Opal 690) incubation for 10 min. MWT was performed at each cycle of staining to remove the Ab TSA complex with AR solution (pH 9 or 6). At last, all slides were counterstained with DAPI for 5 min and enclosed in ProLong Diamond Antifade Mountant (Thermofisher). The slides were scanned using the PerkinElmer Vectra 3 System and the multispectral images obtained were unmixed using spectral libraries previously built from images stained for each fluorophore (monoplex), using the inForm Advanced Image Analysis software (inForm 2.3.0 PerkinElmer). A selection of representative multispectral images belonging to different samples was used to train the inForm software for tissue segmentation, cell segmentation, and phenotyping, and finally, the settings applied to the training images were saved within an algorithm allowing batch analysis of all the tissue slides. We designed two different 7-plex panels defined as phenotype and functional panels, which were used on 2 sequential slides in order to characterize the immune microenvironment of pre-cancer lesions of the lung, including (in)activated cells, (in)activated immune pathways and immune response type. The phenotype panel included CD3, CD8, FoxP3, CD68, Neutrophil elastase (NE), DAPI, and Cytokeratin (CK) and the functional panel included: CD3, PD-L1, PD1, Ki67, CD137, DAPI, and CK.

9) Spatial Statistics

We performed first- and second-order spatial analysis of multispectral imaging data, which enables a high-definition characterization of the microenvironment architecture. First, we reconstructed whole slides, rather than separate analysis of each image that introduces edge effects and leads to loss of information. We calculated immune cell densities as the number of positive cells per unit of tissue surface area (mm2). Based on the tissue categorization performed with the inForm software, the stroma and the epithelium compartments were annotated on the images, enabling densities and spatial distribution to be calculated individually for the stromal and epithelial tissue category. To compare the spatial localization of different immune cell types, we calculated the distances to the nearest neighbors and their distribution implementing edge corrections, G(r). The function G(r) is the cumulative distribution of the distance from a typical random cell X to its nearest cell Y, where the argument r is the radius of the area in which G(r) is evaluated. Deviations from the empirical and the theoretical G(r) function indicate clustered and dispersed patterns.

7) Statistics

The R statistical software (v 3.3.3) was used for statistical analyses and graphical visualization. The null hypotheses were rejected at p-values lower than 0.05, unless indicated otherwise. When comparing tumor- to normal-tissue gene expression, linear mixed-effects model was used to adjust for the confounding factors smoking history, previous caner, between-patient variability, gender, and age. The Benjamini-Hochberg method^{34,35} was applied for multiple testing correction. Post-hoc multiple testing correction was applied for pairwise comparison using Dunn's test.

Results:

Despite the developments in targeted therapies and immunotherapy, advanced lung cancer remains incurable⁴. There are estimates that US lung cancer deaths could be reduced to more than 70,000 per year by early diagnosis and treatment¹. Recently, the Nelson volume CT screening trials showed a reduction of lung cancer mortality by 26% in men and 39-61% in women⁵. Beyond and prior to its early detection, cancer prevention may significantly reduce cancer burden⁶. It is critical to understand the mechanisms underlying lung carcinogenesis, to decipher the role of the microenvironment in early lesion, in order to move into precision medicine including immunotherapy for cancer prevention². In smokers, a range of successive developmental stages precedes invasive lung squamous cell carcinoma⁷ (SCC), making this cancer a convenient model to mechanistically study how cancer develops. However, the rarity of pre-invasive lesion collections explains the limited knowledge of their molecular and immune profiles⁸. Using gene expression profiling and multispectral imaging, we sought to identify the changes in the tumor and its microenvironment during the successive steps of lung squamous carcinogenesis.

We examined a rare dataset of nine morphological stages of lung squamous carcinogenesis, consisting of 122 carefully annotated biopsies from 77 patients (data not shown). Using gene expression profiling, we first identified 7739 genes associated with the nine histological stages of development (linear mixed-effects model, FDR<0.001). Four distinct and successive molecular steps of progression were revealed by semi-supervised hierarchical clustering of the selected genes (data not shown). The first step included normal non-fluorescent and fluorescent biopsies as well as hyperplasia (normal bronchial tissue); the second comprised of metaplasia, mild dysplasia and moderate dysplasia (low-grade); the third combined both severe dysplasia and in situ carcinoma (CIS) (high-grade), while the fourth segregated invasive (SCC) from premalignant lesions (data not shown).

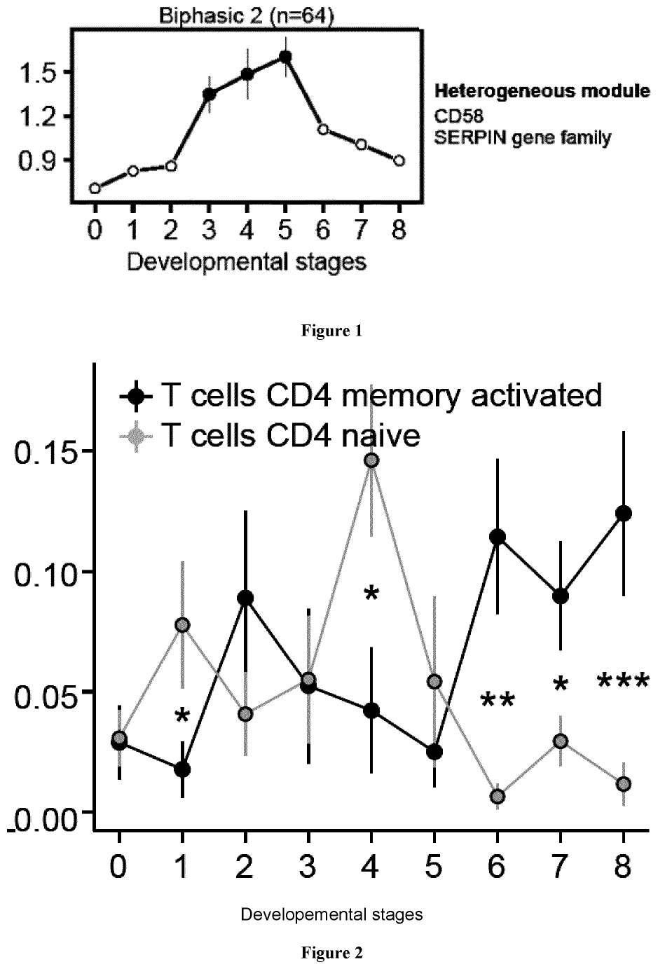

Carcinogenesis has been described as the process of acquiring advantageous biological capabilities, cancer hallmarks, by the abnormal cells⁹. We first isolated modules of genes with specific expression patterns and then searched for significant associations with cancer hallmarks (hypergeometric test, data not shown). Seven evolutionary trajectories of gene expression were discerned by seven gene modules derived from weighted gene co-expression network analysis (WGCNA, data not shown). The two largest modules exhibited linear evolution from normal tissue to cancer, Ascending (n=1848), associated with proliferation and Descending (n=939), linked to genes that are down-regulated in DNA repair (i.e. UV response), suggesting a continuous activation of DNA damage response. A module of 150 genes displayed late expression increase starting from high-grade lesions (High-grade ascending). Interestingly, this module was highly enriched with genes involved in immune response. A set of genes remained unmodified until cancer onset (SCC ascending, n=51). This increase of expression specific to SCC was over-represented by genes involved in epithelial-mesenchymal transition (EMT). The CXCL12-CXCR4 axis known to promote the EMT process, revealed a very low expression of CXCL12, and a significant increase of CXCR4 expression in SCC (data not shown). Two additional modules had biphasic gene expression evolutions, both reaching a peak of expression in low-grade (Biphasic 1, n=164 and Biphasic 2, n=64) (FIG. 1). Here, we found that metabolism regulation had a biphasic trajectory. Specifically, genes involved in fatty acid metabolism, oxidative phosphorylation and citric acid cycle had a transitory increase of expression in low-grade (Biphasic 1).

To analyze the evolutionary trajectory of immune response, we first compiled genes representing specific immune, stromal, and cancer cell types and matched them to each gene module. We confirmed the highest percentage of immune-related genes in the module High-grade ascending along with a significant under-representation in the linearly decreasing module (both p<0.001, Fisher's exact test, data not shown). Cancer-germline antigens were found in the Ascending module at a significantly higher number than expected (FDR<0.05), along with an over-representation trend of genes involved in neutrophil activation (FDR<0.1, data not shown). Both observations suggest immune sensing at the earliest steps of transformation. Markedly, increased gene expression representing activated T cells was detected in high-grade lesions before tumor invasion, with the same pattern as total neutrophils, M1 macrophages, and overall, the myeloid signature.

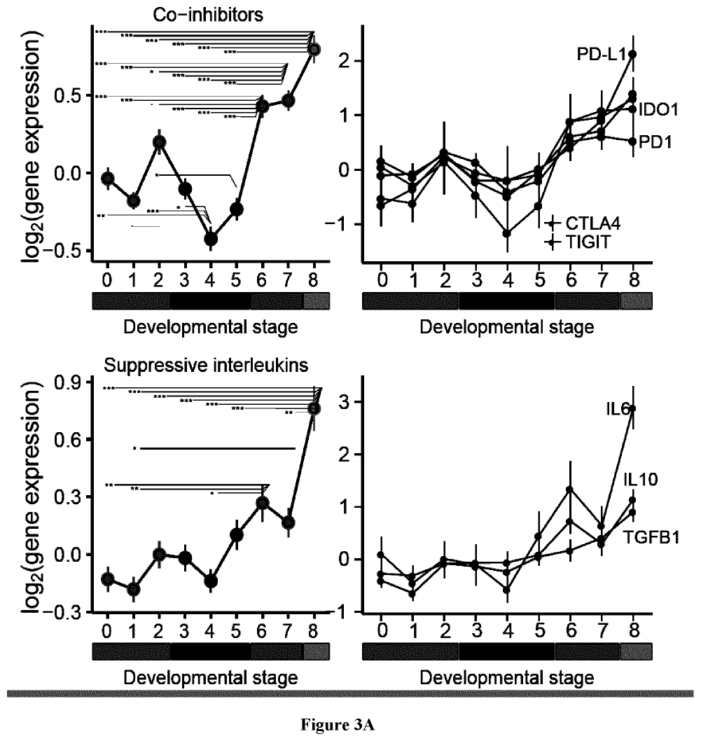

We then estimated the absolute abundance of different immune cell types using a method for deconvolving cell composition of complex tissues from gene expression (data not shown). We confirmed an increase of myeloid-derived cells, neutrophils and macrophage subtypes in high-grade dysplasia (data not shown). Additionally, we observed co-regulation of immune cells from both the innate and adaptive immunity based on correlation of the immune cell abundances (data not shown). Activated T cells (CD4 memory), macrophages (M0), memory B cells, follicular T-helper cells, and dendritic cells followed the same abundance pattern. Interestingly, lesions within the same patient had different immune composition at different developmental stages (data not shown). We also detected a significant shift in the immune status, from resting or naive to activated or memory (data not shown). Resting mast cells were more abundant in the early compared to the late developmental stages, while the activated mast cells followed the opposite pattern (data not shown). A drop of naive B cell abundance was accompanied by an increase of memory B cells. An influx of naive CD4 cells was observed already at the stage of mild dysplasia (stage 4), followed by a sudden decline of naive CD4 abundance and a concurrent increase of activated CD4 memory T cells in the successive stages (FIG. 2).

To further elucidate the immune transition at each molecular step of transformation, we performed functional analysis of the differentially regulated genes in transformed compared to normal tissues. Accounting for smoking history, previous cancer status, and intra-patient variability as confounding factors, we identified Gene Ontology (GO) immune processes enriched among the differentially regulated genes in low-grade, high-grade, and SCC (linear mixed-effects model, FDR<0.05, data not shown). Few immune functions were specifically modulated for low-grade, not only among up-(n=5) but also among down-regulated genes (n=13, e.g. response to TGFβ). Unlike low-grade, a large number of immune functions were enriched only among the up-regulated genes in high-grade (n=148) and SCC (n=240). Strikingly, negative regulation of the immune system was implicated in all developmental stages, in addition to antigen processing and presentation of peptide antigen (data not shown). Nevertheless, in low-grade, the genes associated with negative regulation were significantly down-regulated, while, in high-grade and SCC, they were up-regulated. Therefore, one of the early immune reactions is immune unleashing by down-regulation of the genes that negatively regulate the immune system such as HVEM (TNFRSF14), CD200, CD59, TGFB3, and HLA-G. Reversely, in high-grade and SCC, there was an up-regulation of genes involved in immunosuppression.

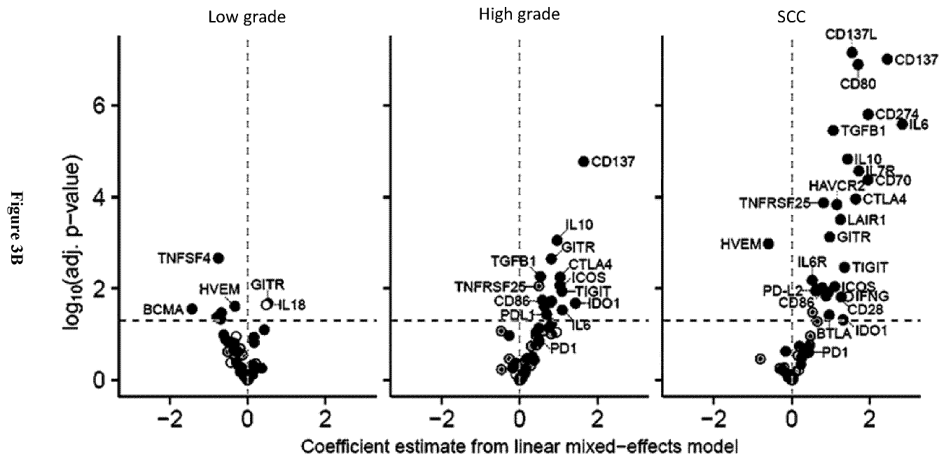

Closer examination of immunomodulatory gene expression revealed that the average expression of co-inhibitory molecules and suppressive interleukins was significantly higher in severe dysplasia (stage 6) and in the succeeding stages (FIG. 3A). Particularly, the expression of PDL1, PD1, IDO1, CTLA4, and TIGIT marked an increase at the transition point to severe dysplasia (stage 6). Similar evolution patterns were observed for the suppressive interleukins, including IL6, IL10, and TGFβ, with elevated expression at the transition from moderate dysplasia (stage 5). Overall, many immunomodulatory molecules had a positive fold change in high-grade dysplasia compared to normal tissue (FIG. 31B). Not only suppressive molecules (IDO1, PDL1, TIGIT, CTLA4, ICOS, IL10, and IL6) but also stimulatory molecules (CD137, GITR, ICOS, CD80, CD86, CD70, CD137L, TNFRSF25) had increased expression in high-grade (linear mixed-effects model, Benjamini and Hochberg (BH)) and to a greater extent at the invasive stage. Collectively, immune escape occurred before tumor invasion as co-inhibitors and suppressive interleukins increased significantly from high-grade stages onwards.

For high-definition characterization of the microenvironment architecture, we used two 7-plex staining panels in FFPE blocks from the same bronchial epithelial lesions, a phenotype panel to determine the nature of the immune cells and a functional panel including PD1, PD-L1, Ki67, and CD137, in addition to CD3, Cytokeratin (CK) and DAPI (n=110 and 106, respectively, data not shown). First, we calculated immune cell densities as the number of positive cells per unit of tissue surface area (mm²), individually for the stromal and epithelial tissue category (data not shown). Overall, we found a relatively large variation in the immune cell densities. However, we observed significant differences among the four developmental stages in the stromal compartment and the same sustained trends in the epithelial compartment (data not shown). CD4 T cells (i.e. CD3⁺CD8⁻) and CD8⁺ lymphocytes both had a transitory increase in high-grade pre-invasive lesions (p<0.01). Consistent with the immune gene expression evolution, myeloid, neutrophil, and macrophage densities increased in high-grade's stroma (p<0.05, FDR<0.1) and epithelium (p<0.1 before BH correction). In accordance with gene expression, PD-L1 (PD-L1⁺CK⁻) densities significantly increased in high-grade lesions and even more in SCC (p<0.05) (data not shown), similarly to CD137, which did not reach statistical significance. Cells with the CD137, PD-L1, and CD3⁺FoxP3⁺ phenotype were rarely found in the epithelium at early developmental stages (i.e. stage 0-5, normal and low-grade).

We next performed second-order spatial statistics and measured distances between each pair of cell phenotypes. We calculated a cross-type cumulative distribution of the nearest neighbor distances, G(r) (data not shown). We expected a potential interaction when two cells were within a distance of 25 μm. By comparison of the observed empirical function G_{X,Y}(r) to the theoretical curve G^{theo}_{X,Y}(r) that shows random sample distribution, we detected segregation among epithelial cells (CK) and CD3, consistently in both panels (p<0.001, FDR<0.1, data not shown). In particular, we observed a lower number of epithelial cells than expected near CD3 cells in high-grade (data not shown). This pattern was observed for all CK⁺ cells in the functional panel, total epithelial cells (all CK⁺), CK⁺PD-L1*, and CK⁺Ki67⁺ (p<0.01, FDR<0.1, data not shown). Therefore, in high-grade, we discerned reconfiguration of the tumor microenvironment compared to the preceding stages of development, manifested by segregation of epithelial cells from CD3 cells.

This report shows that both immune activation and immune suppression occur at pre-invasive stages, which reinforces the use of immunotherapy at the earliest steps of treatment and underlines its potential role in chemopreventive approaches. The prognostic impact of immune infiltrates has been demonstrated in various cancer types¹⁰⁻¹² from early stage¹³, including lung cancer¹⁴ from stage I¹⁵. Tumor intrinsic factors modestly contributed to the risk of carcinogenesis¹⁶, as compared to extrinsic carcinogen¹⁶ or dysregulation of the immune microenvironment¹⁷. We previously showed that the tumor microenvironment was a critical determinant of dissemination to distant metastasis¹⁸ and of metastatic tumor development, where tumor evolution could be traced back to immune escaping clones¹⁷. These findings could also apply to the pre-malignant transformation and the initiation of carcinoma. Furthermore, major clinical benefit of checkpoint immunotherapy was obtained in various settings of cancer treatment. In non-small cell lung cancer (NSCLC), checkpoint inhibitors are now a standard of care as first-line^{19,20} and second-line treatment options^{21,22,23} for advanced disease and as maintenance after curative chemo-radiation of locally advanced stages²⁴. However, up to now, the best opportunity to cure lung cancer patients is early intervention. The positive results of immune checkpoint blockade therapy in adjuvant setting for melanoma²⁵ and in neoadjuvant setting for lung cancer²⁶ fortify the importance of using immunotherapy in the early steps of treatment strategies.

Our study delineated the molecular pathways involved in the four steps of lung squamous cell carcinogenesis (data not shown), whereby the earliest molecular changes affected proliferation and metabolism. A transient influx of naive T cell was observed in low-grade, a pattern previously described for miRNA expression in a subset of the same preneoplastic lesions²¹. Collectively, the immune transition unfolds as follows 1) immune sensing and immune unleashing are induced at the earliest step of transformation; 2) continual cell proliferation fosters accumulation of somatic mutations mounting an anti-tumor immune response and, correspondingly, 3) triggering inherent immune suppression mechanisms already in high-grade pre-cancer. Historically, studies have shown that the risk of cancer progression is much higher in high-grade (32-87%) compared to low-grade lesions (2-9%)²⁸⁻³⁰. Altogether, our results urge to assess the role of immunotherapy and chemoprevention in high-risk individuals for lung cancer.

### REFERENCES

Throughout this application, various references describe the state of the art to which this invention pertains. The disclosures of these references are hereby incorporated by reference into the present disclosure.
1. Goldberg, S. W., Mulshine, J. L., Hagstrom, D. & Pyenson, B. S. An actuarial approach to comparing early stage and late stage lung cancer mortality and survival. Popul. Health Manag. 13, 33-46 (2010).
2. Kensler, T. W. et al. Transforming Cancer Prevention through Precision Medicine and Immune-oncology. Cancer Prev. Res. Phila. Pa. 9, 2-10 (2016).
3. Bindea, G. et al. Spatiotemporal dynamics of intratumoral immune cells reveal the immune landscape in human cancer. Immunity 39, 782-795 (2013).
4. Herbst, R. S., Morgensztern, D. & Boshoff, C. The biology and management of non-small cell lung cancer. Nature 553, 446-454 (2018).
5. De Koning, H., Van Der Aalst, C., Ten Haaf, K. & Oudkerk, M. PLO2.05 Effects of Volume CT Lung Cancer Screening: Mortality Results of the NELSON Randomised-Controlled Population Based Trial. J. Thorac. Oncol. 13, S185 (2018).
6. Umar, A., Dunn, B. K. & Greenwald, P. Future directions in cancer prevention. Nat. Rev. Cancer 12, 835-848 (2012).
7. Slaughter, D. P., Southwick, H. W. & Smejkal, W. Field cancerization in oral stratified squamous epithelium; clinical implications of multicentric origin. Cancer 6, 963-968 (1953).
8. Kerr, K. M. Pulmonary preinvasive neoplasia. J. Clin. Pathol. 54, 257-271 (2001).
9. Hanahan, D. & Weinberg, R. A. Hallmarks of cancer: the next generation. Cell 144, 646-674 (2011).
10. Galon, J. Type, Density, and Location of Immune Cells Within Human Colorectal Tumors Predict Clinical Outcome. Science 313, 1960-1964 (2006).
11. Galon, J., Angell, H. K., Bedognetti, D. & Marincola, F. M. The Continuum of Cancer Immunosurveillance: Prognostic, Predictive, and Mechanistic Signatures. Immunity 39, 11-26 (2013).
12. Pages, F. et al. International validation of the consensus Immunoscore for the classification of colon cancer: a prognostic and accuracy study. The Lancet 391, 2128-2139 (2018).
13. Pages, F. et al. In Situ Cytotoxic and Memory T Cells Predict Outcome in Patients With Early-Stage Colorectal Cancer. J. Clin. Oncol. 27, 5944-5951 (2009).
14. Fridman, W. H., Pages, F., Sautes-Fridman, C. & Galon, J. The immune contexture in human tumours: impact on clinical outcome. Nat. Rev. Cancer 12, 298-306 (2012).
15. Gentles, A. J. et al. The prognostic landscape of genes and infiltrating immune cells across human cancers. Nat. Med. 21, 938-945 (2015).
16. Wu, S., Powers, S., Zhu, W. & Hannun, Y. A. Substantial contribution of extrinsic risk factors to cancer development. Nature 529, 43-47 (2016).
17. Angelova, M. et al. Evolution of Metastases in Space and Time under Immune Selection. Cell 175, 751-765.e16 (2018).
18. Mlecnik, B. et al. The tumor microenvironment and Immunoscore are critical determinants of dissemination to distant metastasis. Sci. Transl. Med. 8, 327ra26 (2016).
19. Gandhi, L. et al. Pembrolizumab plus Chemotherapy in Metastatic Non-Small-Cell Lung Cancer. N. Engl. J. Med. 378, 2078-2092 (2018).
20. Reck, M. et al. Pembrolizumab versus Chemotherapy for PD-L1-Positive Non-Small-Cell Lung Cancer. N. Engl. J. Med. 375, 1823-1833 (2016).
21. Brahmer, J. et al. Nivolumab versus Docetaxel in Advanced Squamous-Cell Non-Small-Cell Lung Cancer. N. Engl. J. Med. 373, 123-135 (2015).
22. Borghaei, H. et al. Nivolumab versus Docetaxel in Advanced Nonsquamous Non-Small-Cell Lung Cancer. N. Engl. J. Med. 373, 1627-1639 (2015).
23. Herbst, R. S. et al. Pembrolizumab versus docetaxel for previously treated, PD-L1-positive, advanced non-small-cell lung cancer (KEYNOTE-010): a randomised controlled trial. Lancet Lond. Engl. 387, 1540-1550 (2016).
24. Antonia, S. J. et al. Overall Survival with Durvalumab after Chemoradiotherapy in Stage III NSCLC. N. Engl. J. Med. (2018). doi:10.1056/NEJMoa1809697
25. Eggermont, A. M. M. et al. Adjuvant Pembrolizumab versus Placebo in Resected Stage III Melanoma. N. Engl. J. Med. 378, 1789-1801 (2018).
26. Forde, P. M. et al. Neoadjuvant PD-1 Blockade in Resectable Lung Cancer. N. Engl. J. Med. 378, 1976-1986 (2018).
27. Mascaux, C. et al. Evolution of microRNA expression during human bronchial squamous carcinogenesis. Eur. Respir. J. 33, 352-359 (2009).
28. Bota, S. et al. Follow-up of Bronchial Precancerous Lesions and Carcinoma in Situ Using Fluorescence Endoscopy. Am. J. Respir. Crit. Care Med. 164, 1688-1693 (2001).
29. Breuer, R. H. et al. The Natural Course of Preneoplastic Lesions in Bronchial Epithelium. 8
30. Venmans, B. J. W., van Boxem, T. J. M., Smit, E. F., Postmus, P. E. & Sutedja, T. G. Outcome of Bronchial Carcinoma In Situ. Chest 117, 1572-1576 (2000).
31. Dobbin, K., Shih, J. H. & Simon, R. Questions and Answers on Design of Dual-Label Microarrays for Identifying Differentially Expressed Genes. JNCI J. Natl. Cancer Inst. 95, 1362-1369 (2003).
32. Tumours of the Lung, Pleura, Thymus and Heart. in Pathology & Genetics (eds. Travis W. D., Brambilla E., Muller-Hermelink H. K. & Harris C.C.) (IARC Press: Lyon, 2004).
33. Kerr, M. K., Martin, M. & Churchill, G. A. Analysis of variance for gene expression microarray data. J. Comput. Biol. J. Comput. Mol. Cell Biol. 7, 819-837 (2000).
34. Qian, H.-R. & Huang, S. Comparison of false discovery rate methods in identifying genes with differential expression. Genomics 86, 495-503 (2005).
35. Ge, Y., Sealfon, S. C. & Speed, T. P. Some step-down procedures controlling the false discovery rate under dependence. Stat. Sin. 18, 881-904 (2008).
36. Langfelder, P. & Horvath, S. WGCNA: an R package for weighted correlation network analysis. BMC Bioinformatics 9, 559 (2008).
37. Liberzon, A. et al. The Molecular Signatures Database Hallmark Gene Set Collection. Cell Syst. 1, 417-425 (2015).
38. Yu, G., Wang, L.-G., Han, Y. & He, Q.-Y. clusterProfiler: an R Package for Comparing Biological Themes Among Gene Clusters. OMICS J. Integr. Biol. 16, 284-287 (2012).
39. Subramanian, A. et al. Gene set enrichment analysis: A knowledge-based approach for interpreting genome-wide expression profiles. Proc. Natl. Acad. Sci. 102, 15545-15550 (2005).
40. Angelova, M. et al. Characterization of the immunophenotypes and antigenomes of colorectal cancers reveals distinct tumor escape mechanisms and novel targets for immunotherapy. Genome Biol. 16, 64 (2015).
41. Newman, A. M. et al. Robust enumeration of cell subsets from tissue expression profiles. Nat. Methods 12, 453-457 (2015).

1. A method for determining whether a subject having a premalignant lesion is at risk of having a cancer comprising determining the level of at least one immune marker in a biological sample obtained from the subject and wherein the expression level of the immune marker correlates with the risk of having cancer.

2. The method of claim 1 wherein the cancer results from polygenic or multifactorial phenotypes.

3. The method of claim 1 wherein the cancer is a lung cancer.

4. The method of claim 1 wherein the sample is a body fluid sample or a tissue sample.

5. The method of claim 1 wherein the premalignant lesion is a low grade dysplasia, the at least one immune marker is a CD58 and/or a SERPIN and the expression level of the at least one immune marker correlates with the risk of having cancer.

6. The method of claim 5 wherein the premalignant lesion is a low grade bronchial dysplasia, the at least one immune marker is a CD58 and/or a SERPIN and the expression level of the at least one immune marker correlates with the risk of having lung cancer.

7. The method of claim 1 wherein the at least one immune marker comprises CD4 naive T cells and wherein the expression level of the at least one immune marker correlates with the risk of having cancer.

8. The method of claim 7, wherein the low grade dysplasia is a low grade bronchial dysplasia, the at least one immune marker comprises CD4 naive T cells and the expression level of the at least one immune marker correlates with the risk of having lung cancer.

9. The method of claim 1, wherein the at least one immune marker is selected from the group consisting of TNFRSF18 (GITR), IL18, TNFRSF14 (HVEM), TNFSF4, and TNFRSF17 (BCMA).

10. The method of claim 9 wherein the at least one immune marker is selected from the group consisting of TNFRSF18 (GITR), IL18, TNFRSF14 (HVEM), TNFSF4, and TNFRSF17 (BCMA).

11. The method of claim 1, wherein the at least one immune marker is selected from the group consisting of co-inhibitory molecules, co-stimulatory molecules, immunosuppressive interleukins and immunostimulatory interleukins.

12. The method of claim 11, wherein the premalignant lesion is a high grade bronchial dysplasia and the expression level of the at least one immune marker correlates with the risk of having lung cancer.

13. The method of claim 11 wherein the immune marker is:
a co-stimulatory molecule selected from the group consisting of CD137, GITR, ICOS, TNFRSF25 and CD86; or
a co-inhibitory molecule selected from the group consisting of PDL1, PD1, IDO1, CTLA4, and TIGIT; or
an immunostimulatory interleukin selected from the group consisting of IL-18 and IFNG; or
an immunosuppressive interleukin selected from the group consisting of IL6, IL10, and TGFβ.

14. (canceled)

15. (canceled)

16. (canceled)

17. The method of claim 1 wherein the step of determining includes detecting the presence of or measuring the amount of messenger RNA (mRNA) transcribed from genomic DNA encoding proteins which are specifically produced by cells from the immune system, or includes detecting the presence of or measuring the amount of proteins expressed by a cell or released in a soluble form.

18. The method of claim 1 wherein the expression level of 2, 3, 4, 5, 6, 7, 8, 9, 10, 11, 12, 13, 14 or 15 immune markers is determined.

19. The method of claim 18, wherein a score which is a composite of the expression level of the immune markers is determined and compared to the predetermined reference value, and wherein a difference between said score and said predetermined reference value is indicative of whether or not the subject is at risk of having cancer.

20. The method of claim 18 which comprises a) quantifying the expression level of a plurality of immune markers in the sample; b) implementing an algorithm on data comprising the quantified plurality of immune markers so as to obtain an algorithm output; and c) determining the probability that the subject will develop a cancer from the algorithm output of step b).

21. A method for the prophylactic treatment of cancer in a subject having at least one premalignant lesion comprising administering to the subject a therapeutically effective amount of at least one chemopreventive agent.

22. The method of claim 21 wherein the subject has been considered as being at risk of having cancer by the method of claim 1.

23. The method of claim 21 wherein the chemopreventive agent is an immune checkpoint inhibitor.

24. The method of claim 23 wherein the immune checkpoint inhibitor is selected from the group consisting of PD-1 antagonists, PD-L1 antagonists, PD-L2 antagonists, CTLA-4 antagonists, VISTA antagonists, TIM-3 antagonists, LAG-3 antagonists, GITR antagonists, IDO antagonists, KIR2D antagonists, A2AR antagonists, B7-H3 antagonists, B7-H4 antagonists, and BTLA antagonists.

25. The method of claim 21 wherein the chemopreventive agent is an inhibitor of an immunosuppressive cytokine.

26. The method of claim 25 wherein the immunosuppressive cytokine is IL6, IL10, or TGFβ.

27. The method of claim 21 wherein the chemopreventive agent is a vaccine against an immune checkpoint inhibitor or a suppressive cytokine or suppressive protein.

28. The method of claim 27 wherein the vaccine against the immune checkpoint inhibitor includes proteins or peptides of PD-1, PD-L1, PD-L2 CTLA-4, VISTA, TIM-3, LAG-3, GITR, IDO, KIR2D, A2AR, B7-H3, B7-H4, and BTLA.

29. The method of claim 27 wherein the vaccine is against IL6, IL10 or TGFβ.

30. The method of claim 21 wherein the chemopreventive agent is administered by systemic route to the subject on or by local route in the premalignant lesion.
